(19) United States
(12) Patent Application Publication
(10) Pub. No.: US 2014/0109656 A1
(43) Pub. Date: Apr. 24, 2014

(54) Apparatus for Measurement of Liquid Oil Products

(71) Applicants: Yury Tronik, Serpukhov (RU); Peter Mouler, Irvine, CA (US); Andrei Kaloshin, Moscow (RU)

(72) Inventors: Yury Tronik, Serpukhov (RU); Peter Mouler, Irvine, CA (US); Andrei Kaloshin, Moscow (RU)

(73) Assignee: ARGOSY TECHNOLOGIES, Palm Desert, CA (US)

(21) Appl. No.: 13/659,387

(22) Filed: Oct. 24, 2012

(51) Int. Cl. G01N 33/28

(52) U.S. Cl. 73/53.05

(57) ABSTRACT

An apparatus for measurement of liquid oil products has a local control unit; a grounding element; an electric pump aggregate; a filter; a gas separator; an electromagnetically operated valve; an indication and control unit; a sensor for sensing temperature of liquid oil product; a mass flow meter located between an outlet of the gas separator and an input of the electromagnetically operated valve and connected with the indication and control block; and a device for determining a water quantity in the oil product connected with the indication and control block.

### BACKGROUND OF THE INVENTION

The present invention generally relates to apparatuses which are used for dosing of liquid oil products.

More particularly, it relates to liquid measurement apparatuses which can be used for fuel transportation vehicles, fuel dispensing stations for vehicles, oil storage facilities, oil pumping facilities, and terminals for filling and draining oil and oil processing products.

RU 63346 discloses an equipment complex for draining and filling of oil products into vehicle and railroad containers. It includes a filling stand, a filter, a gas separator, a valve, a density measuring unit, a thermal sensor, a remote control stand, formed as a single level module with a frame, which supports a hydraulic block including a grounding device, a terminals box, a local control stand, an electric pump aggregate, a composite filter-gas separator unit, a liquid oil product counter with a pulse sensor, a valve with an electromagnetic control and a hydraulic lock, with an outlet to hydraulic filling consoles for upper and lower filling. The complex has a low reliability, has a complicated and large structure, and allow a high error in determination of mass of a dose.

Another automated measuring complex for draining and filling of oil products into vehicle and railroad containers is disclosed in RU 63793. It include a filing stand, a filter, a gas separator, a liquid counter with a pulse sensor, a valve, a density measuring unit, a thermal sensor, a local and remote control stand formed as a single level module with a frame which supports a hydraulic block including an ultrasound counter, a grounding element, an electric pump aggregates composite filter-gas separator unit, an indication and control block with an electronic adjustment, a valve with an electromagnetic control and a hydraulic lock, with an outlet to hermetic filling consoles for upper and lower filling. This system is also characterized by a high error in measurements of volume and determination of mass of a liquid oil product due to an indirect method of dynamic measurements, and is lacks a capability of determination of water quantity in oil products, with which it deals.

### SUMMARY OF THE INVENTION

Accordingly it is an object of the present invention to provide a new apparatus for measurement and dosing of liquid oil products, which eliminates the disadvantages of the prior art and contains further improvements.

In keeping with these objects and with others which will become apparent hereinafter, one feature of the present invention resides, briefly stated, in an apparatus for measurement of liquid oil products, which has a local control unit; a grounding means; an electric pump aggregate; a filter; a gas separator; an electromagnetically operated valve; an indication and control unit; a sensor for sensing temperature of liquid oil product; a Coriolis type mass flow meter located between an outlet of said gas separator and an input of said electromagnetically operated valve and connected with said indication and control unit, and a device for determining a quantity of water in a liquid oil product.

In accordance with another feature of the present invention in the inventive apparatus for measurement of liquids the mass flow meter is connected with the indication and control unit via an interface.

In the apparatus for measurement of liquids in accordance with the present invention, the mass flow meter is located vertically and configured so that a liquid oil product to be measured is supplied into the mass flow meter from below upwardly.

In accordance with a further feature of the present invention, the apparatus for measurement of liquid oil products further comprises means forming an inlet into the apparatus, and a return valve located at the inlet into the apparatus.

In accordance with still a further feature of the present invention, the device for determining a water quantity in an oil product is located between the gas separator and a mass flow meter and connected with the indication and control unit.

The apparatus for measurement of liquid oil products in accordance with the present invention has a higher accuracy of measurement and a higher reliability of the operation, and has a simpler and more compact construction than the existing apparatuses of this type.

The apparatus provides measurements of a quantity of oil products in volume and mass units by a straight dynamic process and includes measurements of five parameters, in particular mass, density and volume with the mass flow meter, preferably of a Coriolis type, temperature of the oil products by the temperature sensor, and a water quantity in a dispensed dose of the oil product by the device for determining a water quantity in the oil product.

The novel features of the present invention are set forth in particular in the appended claims. The invention itself however will be best understood from the following description of the preferred embodiments, which is accompanied by the following drawings.

### BRIEF DESCRIPTION OF THE DRAWINGS

FIG. 1 is a side view of an apparatus for measurement and dosing of oil products in accordance with the present invention; and

FIG. 2 is a plan view of the inventive apparatus for measurement and dosing of oil products.

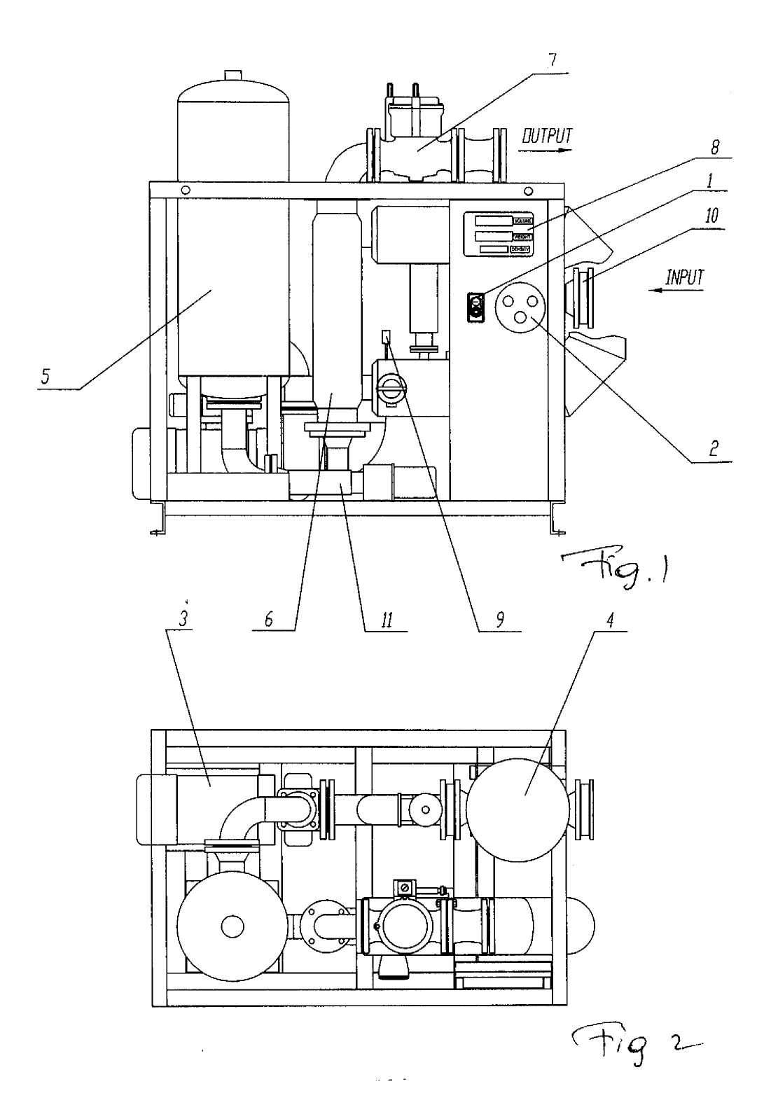

### DESCRIPTION OF THE PREFERRED EMBODIMENTS

An apparatus for measurement and dosing of liquid oil products in accordance with the present invention has local control stand 1, a grounding unit 2, an electric pump aggregate 3, a filter 4, a gas separator 5, a mass flow meter 6, a valve with an electromagnetic control 7, an indication and control block 8, a sensor of temperature of a liquid 9, a return valve 10, and a device for determining a water quantity in an oil product 11.

The local control post 1 is used for turning on and an emergency turning off of the apparatus. The grounding unit 2 connects a housing of a container to a grounding circuit. The electric pump aggregate supplies liquid oil product from a reservoir. The gas separator removes air or dissolved gasses from liquid oil product before it is supplied to the mass flow meter.

The mass flow meter is of a Coriolis type. The utilized mass flow meter can be for example of Micro Motion models CMF, D, DS, F of “Emerson Process Management, Micro Motion Inc.” It includes a sensor which directly measures a flow rate, density of oil product and temperature of sensor pipes and a converter which converts sensor information into output signals. During movement of liquid through the sensor, a Coriolis effect is produced which leads to a Coriolis force causing a bending of the sensor pipes, which is proportional to a mass flow rate of the liquid oil product and has a phase shift due to time difference between signals of inlet and outlet detector

The time difference between the signals is proportional to the mass flow which thereby can be determined therefrom.

The valve with the electromagnetic control 7 regulates a flow rate of oil products during dispensing of a dose and has a large cross section channel and a small cross section channel which are closed when the apparatus does not dispense liquid oil products. In order to avoid a hydraulic surge, first the small cross section channel is opened and a dose of liquid oil product is supplied with a low flow rate. Then the large cross section channel is opened and the liquid oil product is supplied with a higher flow rate. At the end of dispensing of the dose, first the large cross section channel is closed to reduce the flow rate and thereafter the small cross section channel is closed as well, to provide dispensing of liquid oil product with high accuracy in accordance with the required dose.

The indication and control block 8 is an electronic microprocessing device for exhibiting information about a dispensed quantity of liquid oil product and for controlling the units of the apparatus during dispensing of the dose. The temperature sensor 9 measures a temperature of liquid oil product in the dispensed dose to transmit this information to the indication and control block. The return valve 10 prevent return movement of the liquid oil product after finishing the process of dispensing the dose.

The device for determining a quantity of water in the oil product 11 is located between the gas separator and the mass flow meter and connected with the indication and control block. It can be formed, for example, as a device disclosed in our Utility Model document RU 94 344 or as a device disclosed in our U.S. patent application Ser. No. filed on. The device can have a coaxial resonator unit for passing the oil product therethrough and an electrical measuring bridge connected with the resonator. During passage of the oil product through the resonator the frequency characteristic of the resonator varies and frequency characteristic changes are measured and used for determination of a quantity of water in the oil product.

The inventive apparatus for measurement and dosing of liquid oil products operates in the following manner.

After receiving a signal from the system of control and permission command from the local control stand 1 and the grounding unit 2, the indication and control block 8 turns on the electric pump aggregate 3 and the valve with the electromagnetic control 7 with a small cross section, thus preventing hydraulic surges.

The liquid oil product moves with a low flow rate from the receiving pipe through the return valve 10 into the filter 4 where the liquid oil product is cleaned from mechanical impurities. Then the liquid oil product is supplied into the gas separator 5, in which separation from liquid oil product and withdrawal of air and vapors, which could affect the accuracy of measurements.

From the gas separator 5 the liquid oil product is supplied into the water quantity measuring device 11 and then into mass flow meter 6, and then, through the open small cross section channel of the valve 7 into an outlet pipeline. After the whole volume of the liquid oil product is involved into motion, the large cross section channel is opened and the liquid oil product is supplied with nominal flow rate.

At the end of the process of dispensing the dose the large cross section channel of the valve 6 is closed and the flow rate of the apparatus is reduced. At the end of dispensing of the dose the small cross section channel is closed as well, and the operation stops after thereby dispensing of the exactly specified dose.

The indication and control block 8 receives information from the mass flow meter, the temperature sensor, and the device for determining water quantity via interface and exhibits on its indicators the information about a single dispensing of the oil product and stores the information about total quantity of dispensed liquid oil product.

The return valve 10 prevents a return flow of the oil product after the end of dispensing of the dose from the outlet pipeline. The temperature sensor 9 provides measurement of the temperature in the dispensed dose of the oil product and transmits this information to the indication and control block 8. The water quantity measuring device provides measurement of a share of water in the dispensed dose of the oil product and transmits this information to the indication and control block 8 as well. The indication and control block exhibits on its display the information about the quantity of water in the dispensed dose of the oil product.

The invention is not limited to the details shown since various modifications and structural changes are possible without departing from the spirit of the invention.

What is desired to be protected by Letters Patent is set forth in the appended claims:

1. Apparatus for measurement of liquid oil products, comprising
a local control unit; a grounding means; an electric pump aggregate; a filter, a gas separator; an electromagnetically operated valve; an indication and control unit;
a sensor for sensing temperature of liquid oil product; a mass flow meter located between an outlet of said gas separator and an input of said electromagnetically operated valve and connected with said indication and control unit; and a device for determining a quantity of water in the oil product connected with said indication and control unit which displays an information about the determined quantity of water.

2. An apparatus for measurement of liquid oil products as defined in claim 1, wherein said mass flow meter and said device for determining a water quantity in the oil product are connected with said indication and control unit via an interfaces.

3. An apparatus for measurement of liquid oil products as defined in claim 1, wherein said mass flow meter is located vertically and configured so that a liquid oil product to be measured is supplied into said mass flow meter from below upwardly.

4. An apparatus for measurement of liquid products as defined in claim 1, wherein said device for determining a water quantity in the liquid oil product is located between said gas separator and said mass flow meter.

5. An apparatus for measurement of liquid oil products as defined in claim 1, further comprising means forming an inlet into the apparatus, and a return valve located at said inlet into the apparatus,
